(19) United States
(12) Patent Application Publication
(10) Pub. No.: US 2025/0152481 A1
(43) Pub. Date: May 15, 2025

(54) COMPOSITION CONTAINING LIPOSOMES AND BIOSURFACTANTS

(71) Applicant: Evonik Operations GmbH, Essen (DE)

(72) Inventors: Pia Dahlhoff, Fulda (DE); Michael Sacher, Schluechtern-Breitenbach (DE)

(73) Assignee: Evonik Operations GmbH, Essen (DE)

(21) Appl. No.: 18/839,866

(22) PCT Filed: Feb. 20, 2023
(86) PCT No.: PCT/EP2023/054171

§ 371 Date: Aug. 20, 2024

(30) Foreign Application Priority Data
Feb. 24, 2022 (EP) 22158501.1

(51) Int. Cl. A61K 8/14; A61K 8/11; A61K 8/60; A61K 8/63; A61K 8/68; A61K 9/1272; A61K 9/48; A61K 31/133; A61K 31/164; A61K 31/56; A61K 47/26; A61Q 19/00

(52) U.S. Cl. CPC A61K 8/14; A61K 8/11; A61K 8/602; A61K 8/63; A61K 8/68; A61K 9/1272; A61K 9/4808; A61K 31/133; A61K 31/164; A61K 31/56; A61K 47/26; A61Q 19/00; A61K 2800/10; A61K 2800/412; A61K 2800/413

(57) ABSTRACT

A liposomal composition includes at least one phospholipid, at least one biosurfactant, water, and optionally at least one solvent. The at least one solvent is an alcohol with at least three carbon atoms or a polyol. A process encapsulates at least one cosmetic, pharmaceutical and/or nutraceutical active ingredient. A formulation and a capsule include said liposomnal composition.

### FIELD OF THE INVENTION

The invention relates to a liposomal composition comprising biosurfactants and the use of the liposomal composition for encapsulation of at least one cosmetic, pharmaceutical and/or nutraceutical active ingredient.

### PRIOR ART

U.S. Pat. No. 4,902,512 discloses liposomes formed by 2-O-alpha-decenoyl-alpha-L-rhamnopyranosyl-beta-hydroxydecanoyl-beta-hydroxydecanoic acid and 2-O-(2-O-alpha-decenoyl-alpha-rhamnopyranosyl)-alpha-L-rhamnopyranosyl-beta-hydroxydecanoyl-beta-hydroxydecanoic acid.

US2019021338A1 discloses the application of a peptide inside a rhamnolipid liposome to plants, trees and bushes to kill diseases and plant pathogens affecting those plants, trees and bushes.

CN108158997 discloses the use of rhamnolipids for the preparation of membrane materials targeted to tumor cell liposomes is characterized in that said tumor cells are tumor cells expressing rhamnose receptors.

EP2654779 discloses liposomes comprising: a lipid bilayer membrane surrounding an aqueous core, wherein the lipid bilayer comprises:
(a) a nonglycosidic ceramide present in an amount of about 1 wt. % to about 50 wt. %, based on the total weight of the liposome or the lipid bilayer; and
(b) one to five lipids present in an amount of about 50 wt. % to about 99 wt. %, based on the total weight of the liposome or the lipid bilayer.

CN100536817 discloses ceramide nanoliposome preparations, characterized in that the ceramide nanoliposome preparation contains the following components by weight percentage: ceramide 0.1 to ˜5%, phospholipid 0.1 to ˜20%, glycerol 10 to ˜70% and water 25 to ˜80%, EP1138311 discloses pro-liposomally encapsulated preparations obtainable by treating UV light protection filters and/or antioxidants in cosmetically acceptable solvents with lecithins and/or phospholipids.

It is an object of the invention to provide liposomal compositions which allows solubilization of hardly soluble substances with outstanding long-time stability in terms of re-precipitation.

### DESCRIPTION OF THE INVENTION

It was found that, surprisingly, that liposomal compositions comprising phospholipid and biosurfactant solve the problem of the instant invention.

The present invention therefore provides a composition comprising liposomes and biosurfactant as described in more detail in claim 1.

The invention further provides a process for encapsulating at least one cosmetic, pharmaceutical and/or nutraceutical active as described in more detail in claim 11.

One advantage of the present invention is that the compositions according to the instant invention have an outstanding stability.

Another advantage of the present invention is that the compositions according to the instant invention can keep hardly soluble substances in solution.

A further advantage is that precipitation and/or crystallization of for examples actives from liposomes will be prevented.

Another advantage of the present invention is that the bioavailability of actives, especially hardly soluble actives, is enhanced.

Another advantage of the present invention is that the compositions according to the instant invention are easy to formulate into cosmetic, pharmaceutical and nutraceutical applications.

Another advantage of the present invention is that the irritation potential of compounds contained in the compositions according to the instant invention is decreased.

Another advantage of the present invention is that the composition according to the instant invention enables economic and sustainable processing at low temperatures.

Another advantage of the present invention is that the composition according to the instant invention chemically stabilizes actives, for example prevents the degradation of actives.

Another advantage of the present invention is that the composition according to the instant invention enhances compatibility of hardly soluble substances with other formulation ingredients.

Another advantage of the present invention is that the composition according to the instant invention is non-irritant to the skin.

Another advantage of the present invention is that the biosurfactants contained in the composition according to the instant invention stabilize the liposomal structures.

Another advantage of the present invention is that the composition according to the instant invention simplifies the incorporation of hardly soluble substances in pharmaceutical, cosmetic and nutraceutical applications.

The instant invention thus provides a liposomal composition comprising
A) at least one phospholipid, which is at least in part located in the bilayer of the liposomes,
B) at least one biosurfactant,
C) water, and optionally
D) at least one solvent selected from the group of alcohols comprising at least three carbon atoms and polyols.

Within the context of the present invention, “biosurfactants” are understood as meaning all glycolipids produced by fermentation. The term “biosurfactant” also covers glycolipids that are chemically or enzymatically modified after fermentation, as long as structurally a glycolipid remains.

Raw materials for producing the biosurfactants that can be used are carbohydrates, in particular sugars such as e.g. glucose and/or lipophilic carbon sources such as fats, oils, partial glycerides, fatty acids, fatty alcohols, long-chain saturated or unsaturated hydrocarbons.

Where average values are stated hereinbelow, then, unless stated otherwise, these are number-averaged average values.

Unless stated otherwise, percentages are data in percent by weight.

Wherever measurement values are stated hereinbelow, then, unless stated otherwise, these have been determined at a temperature of 25° C. and a pressure of 1013 mbar.

A preferred composition according to the instant invention is characterized in that the at least one phospholipid is selected from phosphatidylcholines, also known as lecithins and often abbreviated as PC, phosphatidylethanolamines, also known as cephalins and often abbreviated as PE, phosphatidic acid also known as phosphatidates and often abbreviated as PA, phosphatidylserines often abbreviated as PS, phosphoinositides, like for example phosphatidylinositol (often abbreviated as PI), phosphatidylinositol phosphate (often abbreviated as PIP), phosphatidylinositol bisphosphate (often abbreviated as PIP2) and phosphatidylinositol trisphosphate (often abbreviated as PIP3), and phosphosphingolipids, like for example ceramide phosphorylcholines (often abbreviated as SPH), ceramide phosphorylethanolamines (often abbreviated as Cer-PE) and ceramide phosphoryllipids, preferably phosphatidylcholines.

Phospholipids are commonly obtained from biological sources, for examples plants and animals.

Industrial common sources are soya, rapeseed, sunflower, chicken eggs, bovine milk and fish eggs.

Preferably the composition according to the instant invention is characterized in that it comprises as component A) phosphatidylcholines, wherein the phosphatidylcholine is preferably derived from plants, preferably from the group of soya, sunflowers, rapeseed, lupines, pea, and beans, with soya and sunflowers being especially preferred and sunflower the most.

The composition according to the instant invention preferably comprises as component B) at least one biosurfactant selected from rhamnolipids, sophorolipids, glucolipids, cellulose lipids, mannosylerythritol lipids and trehalose lipids, preferably rhamnolipids, sophorolipids, mannosylerythritol lipids and glucolipids, more preferably rhamnolipids, sophorolipids and glucolipids, more preferably rhamnolipids and glucolipids, most preferably rhamnolipids.

The biosurfactants can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789 or the documents cited therein. Suitable biosurfactants can be acquired e.g. from Soliance, France.

Preferably, the composition according to the instant invention has, as biosurfactant at least one selected from rhamnolipids, in particular mono-, di- or polyrhamnolipids, glucolipids, in particular mono-, di- or polyglucolipids, and sophorolipids, in particular mono-, di- or polysophorolipids, most preferably rhamnolipids.

The term “rhamnolipids” in the context of the present invention preferably is understood to mean particularly compounds of the general formula (I) and salts thereof,

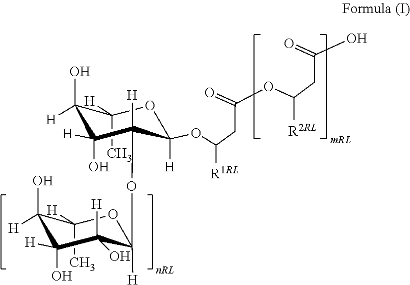

where
mRL=2, 1 or 0,
nRL=1 or 0,
R^{1RL} and R^{2RL}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or triunsaturated alkyl residues, preferably those selected from the group consisting of pentenyl, heptenyl, nonenyl, undecenyl and tridecenyl and (CH₂)ₒ—CH₃ where o=1 to 23, preferably 4 to 12.

If nRL=1, the glycosidic bond between the two rhamnose units is preferably in the α-configuration.

The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where nRL=1.

The term “mono-rhamnolipid” in the context of the present invention is understood to mean compounds of the general formula (I) or salts thereof, where nRL=0.

Distinct rhamnolipids are abbreviated according to the following nomenclature: “diRL-CXCY” are understood to mean di-rhamnolipids of the general formula (I), in which one of the residues R^{1RL} and R^{2RL}=(CH₂)ₒ—CH₃ where o=X-4 and the remaining residue R¹ or R²=(CH₂)ₒ—CH₃ where o=Y-4.

“monoRL-CXCY” are understood to mean mono-rhamnolipids of the general formula (I), in which one of the residues R^{1RL} and R^{2RL}=(CH₂)ₒ—CH₃ where o=X-4 and the remaining residue R^{1RL} or R^{2RL}=(CH₂)ₒ—CH₃ where o=Y-4.

The nomenclature used therefore does not distinguish between “CXCY” and “CYCX”.

For rhamnolipids where mRL=O, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X-3 or Y-3 carbon atoms having Z double bonds.

Methods for preparing the relevant rhamnolipids are disclosed, for example, in EP2786743 and EP2787065.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et aL. in Biotechnol Lett. 2020 June;42(6):997-1002.

Rhamnolipids produced by Pseudomonas aeruginosa are commercially available from Jeneil Biotech Inc., e.g. under the tradename Zonix,from Logos Technologies (technology acquired by Stepan), e.g. under the tradename NatSurFac, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The present invention provides a composition preferably comprising as biosurfactant rhamnolipids, characterized in that the biosurfactant component B) comprises 51% by weight to 95% by weight, preferably 55% by weight to 80% by weight, particularly preferably 60% by weight to 70% by weight, of diRL-C10C10 and where the percentages by weight refer to the sum of all of the rhamnolipids present.

A preferred composition according to the invention is characterized in that the composition comprises as biosurfactant rhamnolipids as described above with a content of 0.5% by weight to 15% by weight, preferably 3% by weight to 12% by weight, particularly preferably 5% by weight to 10% by weight, of diRL-C10C12:1, where the percentages by weight refer to the sum of all of the rhamnolipids present.

A further preferred composition according to the invention is characterized in that the composition comprises as biosurfactant rhamnolipids as described above with a content of 0.5 to 25% by weight, preferably 5% by weight to 15% by weight, particularly preferably 7% by weight to 12% by weight, of diRL-C10C12, where the percentages by weight refer to the sum of all of the rhamnolipids present.

A further preferred composition according to the invention is characterized in that the composition comprises as biosurfactant rhamnolipids as described above with a content of 0.1% by weight to 25% by weight, preferably 2% by weight to 10% by weight, particularly preferably 4% by weight to 8% by weight, of diRL-C8C10, where the percentages by weight refer to the sum total of all rhamnolipids present.

An even further preferred composition according to the invention is characterized in that the composition comprises as biosurfactant rhamnolipids as described above with a content of 0.1% by weight to 5% by weight, preferably 0.5% by weight to 3% by weight, particularly preferably 0.5% by weight to 2% by weight, of monoRL-C8C10 and/or, preferably and 0.1% by weight to 5% by weight, preferably 0.5% by weight to 3% by weight, particularly preferably 0.5% by weight to 2% by weight, of monoRL-C10C10, where the percentages by weight refer to the sum of all of the rhamnolipids present.

The present invention provides a composition alternatively preferably comprising as biosurfactant rhamnolipids, characterized in that the biosurfactant component B) comprises 10% by weight to 50% by weight, preferably 20% by weight to 40% by weight, particularly preferably 25% by weight to 35% by weight, of monoRL-C10C10 and where the percentages by weight refer to the sum of all of the rhamnolipids present.

The alternatively preferred composition according to the invention is preferably characterized in that the composition comprises as biosurfactant rhamnolipids as described above with a content of 10% by weight to 30% by weight, preferably 12% by weight to 25% by weight, particularly preferably 15% by weight to 20% by weight, of diRL-C10C10, where the percentages by weight refer to the sum of all of the rhamnolipids present.

The alternatively preferred composition according to the invention is characterized in that the composition comprises as biosurfactant rhamnolipids as described above with a content of 10% by weight to 30% by weight, preferably 12% by weight to 25% by weight, particularly preferably 15% by weight to 20% by weight, of monoRL-C8C10, where the percentages by weight refer to the sum of all of the rhamnolipids present.

The alternatively preferred composition according to the invention is characterized in that the composition comprises as biosurfactant rhamnolipids as described above with a content of 3% by weight to 25% by weight, preferably 5% by weight to 20% by weight, particularly preferably 10% by weight to 15% by weight, of monoRL-C10C12:1, where the percentages by weight refer to the sum total of all rhamnolipids present.

The alternatively preferred composition according to the invention is characterized in that the composition comprises as biosurfactant rhamnolipids as described above with a content of 1% by weight to 15% by weight, preferably 2% by weight to 10% by weight, particularly preferably 3% by weight to 8% by weight, of diRL-C10C12, where the percentages by weight refer to the sum of all of the rhamnolipids present.

In the context of the present invention, the term “sophorolipids” preferably is understood as meaning compounds of the general formulae (Ila) and (Ilb) and salts thereof

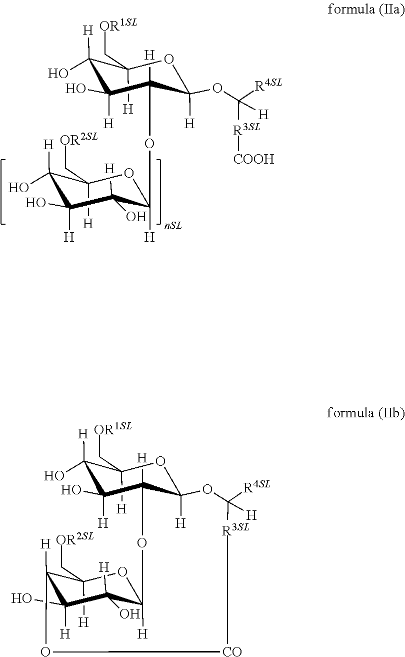

where
R^{1SL}=H or CO—CH₃,
R^{2SL}=H or CO—CH₃,
R^{3SL}=a divalent organic moiety which comprises 6 to 32 carbon atoms and which is unsubstituted or substituted by hydroxyl functions, is unbranched and optionally comprises one to three double or triple bonds,
R^{4SL}=H, CH₃ or a monovalent organic radical which comprises 2 to 10 carbon atoms and which is unsubstituted or substituted by hydroxyl functions, which is unbranched and which optionally comprises one to three double or triple bonds, and
nSL=1 or 0.

Sophorolipids may be used in accordance with the invention in their acid form or their lactone form.

Preferred compositions according to the instant invention comprise a sophorolipid in which the ratio by weight of lactone form to acid form is in the range of 20:80 to 80:20, especially preferably in the ranges of 30:70 to 40:60.

To determine the content of sophorolipids in the acid or lactone form in a formulation, refer to EP1411111B1, page 8, paragraph [0053].

In connection with the present invention, the term “glucolipids” preferably is understood as meaning compounds of the general formula (III) and salts thereof,

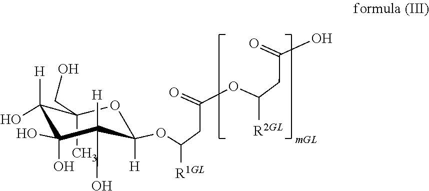

where
mGL=1 or 0,
R^{1GL} and R^{2GL}=independently of one another identical or different organic radical having 2 to 24 carbon atoms, in particular optionally branched, optionally substituted, in particular hydroxy-substituted, optionally unsaturated, in particular optionally mono-, di- or triunsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12.
Distinct glucolipids are abbreviated according to the following nomenclature: “GL-CXCY” is understood as meaning glucolipids of the general formula (III) in which one of the radicals R^{1GL} and R^{2GL}=(CH₂)ₒ—CH₃ where o=X-4 and the remaining radical R^{1GL} or R^{2GL}=(CH₂)ₒ—CH₃ where o=Y-4.

The nomenclature used thus does not differentiate between “CXCY” and “CYCX”.

If one of the aforementioned indices X and/or Y is provided with “:Z”, then this means that the respective radical R^{1GL} and/or R^{2GL}=an unbranched, unsubstituted hydrocarbon radical with X-3 or Y-3 carbon atoms having Z double bonds.

Methods for production of glucolipids can be carried out as described in WO2019154970.

It is preferred that a composition according to the instant invention contains component D), the at least one solvent, especially in the case of that component B), the biosurfactant is selected from rhamnolipids.

A preferred composition according to the instant invention is characterized in that the at least one solvent in component D) is selected from alcohols of the group consisting of alkanols and alkenols.

Preferred alkanols and alkenols have preferably 3 to 30 carbon atoms. They can be straight chained like for example propanol, butanol, pentanol, hexanol, heptanol, octanol, 1-dodecanol, 1-tetradecanol, 1-hexadecanol, 1-octadecanol, 1-docosanol, cis-9-Hexadecen-1-ol, cis-9-Octadecen-1-ol, trans-9-Octadecen-1-ol, cis,cis-9,12-Octadecadien-1-ol, 6,9,12-Octadecatrien-1-ol, or branched chained like for example 16-methylheptadecan-1-ol, 3-methyl-1-butanol and octyldodecanol, with straight chained alkanols being preferred.

A preferred composition according to the instant invention is characterized in that the at least one solvent is selected from diols and triols, preferably glycols with preferably 3 to 30 carbon atoms, more preferably from the group consisting of propanediol, butanediol, preferably 1,3-butanediol, pentanediol, preferably 1,2-pentanediol, hexanediol, preferably 1,2-hexanediol, octanediol, preferably 1,2-octanediol, decanediol, preferably 1,2-decanediol, and glycerol, with glycerol being especially preferred.

The compositions according to the instant invention can be used advantageously for the encapsulation and/or stabilisation of active ingredients. Thus, it is preferred in the instant invention, if the composition according to the instant invention comprises
E) at least one cosmetic, pharmaceutical and/or nutraceutical active ingredient, preferably with a water solubility at 25° C. and 1 bar of less than 1, preferably less than 0.1, mg/ml.

Preferably component E) of the composition according to the instant invention is selected from cosmetic active ingredients.

The cosmetic, pharmaceutical and/or nutraceutical active ingredient comprised in the composition according to the instant invention is preferably contained at least in parts in the bilayer of the liposomes contained in the composition according to the instant invention.

Preferably comprised pharmaceutical and/or nutraceutical active ingredients in this context are selected from glycogen, ceramides, sphingoid bases, sterols, peptides, amino acids, phenols, polyphenols, vitamins, extracts from plants or algae and cosmetic oils, with ceramides, sphingoid bases and sterols being especially preferred.

All ceramides, sphingoid bases, sterols, peptides, polyphenols, vitamins, plant extracts and cosmetic oils, can be comprised in the composition according to the instant invention.

Preferred ceramides comprised in the composition according to the instant invention are selected from the group comprising, preferably consisting of, ceramide NP, ceramide AP, ceramide EOP, ceramide NDS, ceramide ADS, ceramide EODS, ceramide NS, ceramide AS, ceramide EOS, ceramide NH, ceramide AH and ceramide EOH, preferably selected from the group comprising, preferably consisting of, ceramide NP, ceramide AP, ceramide NS, ceramide EOP and ceramide EOS.

The ceramide nomenclature is commonly used and is described in more detail in Farwick et al.

Developments in Ceramide Identification, Synthesis, Function and Nomenclature, Cosmet Toil 2009; 124: 63-72.

Preferred sphingoid bases comprised in the composition according to the instant invention are selected from the group comprising, preferably consisting of, sphingosine, sphinganine, 6-hydroxysphingosine and phytosphingosine.

Preferred sterols comprised in the composition according to the instant invention are selected from the group comprising, preferably consisting of, cholesterol, 7-dehydrocholesterol, potassium cholesterol sulfate, cholesteryl succinate, 25-hydroxy-7-dehydrocholesterol, ergosterol, fucosterol, hopanoids, hydroxysteroid, phytosterols like for example campesterol, sitosterol,and stigmasterol, steroids and zoosterols, with cholesterol being especially preferred.

Preferred peptides comprised in the composition according to the instant invention are selected from the group comprising, preferably consisting of, palmitoyl hexapeptide-12, palmitoyl oligopeptide, palmitoyl pentapeptide-3, palmitoyl tetrapeptide-7, palmitoyl tripeptide-1, collagen, elastin, epidermal growth factor (EGF), epitalon and fibronectin.

Preferred amino acids comprised in the composition according to the instant invention are selected from the group comprising, preferably consisting of, proteinogenic amino acids, preferably alanine, valine, isoleucine, leucine, methionine, phenylalanine, tyrosine and tryptophan.

Preferred phenols and polyphenols comprised in the composition according to the instant invention are selected from the group comprising, preferably consisting of, resveratrol, quercetin, rutin, ellagic acid, apigenin, phloretin, caftaric acid, caffeic acid phenylethyl ester, pterostilbene, luteolin, fisetin, honokiol, ferulic acid and sinapic acid.

Preferred vitamins comprised in the composition according to the instant invention are selected from the group comprising, preferably consisting of, coenzyme q10 (ubiquinone), biotin, vitamin A, retin-A, retinoids, retinol, retinaldehyde, retinol palmitate, retinyl ascorbate, retinyl palmitate, retinyl retinoate, vitamin D, cholecalciferol, ergocalciferol, vitamin E, preferably alpha-, beta-, gamma-, and delta-tocopherol, tocopherol, tocopherol acetate, tocopheryl acetate, tocopheryl lineolate, tocopheryl linoleate, tocopheryl linoleate/oleate, tocopheryl nicotinate, tocopheryl succinate, tocotrienols, vitamin K and vitamin C, preferably ascorbyl palmitate.

Preferred extracts from plants or algae comprised in the composition according to the instant invention are selected from the group comprising, preferably consisting of, aloe (especially vera) extract, aesculus hippocastanum extract, Calendula officinalis flower extract, centella asiatica extract, bakuchiol, ascophyllum nodosum extract, acemannan, coleus barbatus extract, cranberry seed extract, Ginkgo biloba leaf extract, ginseng extract, grapefruit peel extract, green tea extract, Hordeum vulgare extract, horse chestnut extract, Oryza sativa (rice) extract, pectin, Polygonum cuspidatum root extract, pomegranate extract, spirulina extract, squalene, st. john's wort extract, willow bark extract, capsaicin, capsicum extracts, Glycyrrhiza glabra extract, madecassoside, Rosmarinus officinalis extract, ruscogenine, carotinoids, preferably astaxanthin, ß-carotin, canthaxanthin, capsanthin, capsorubin, cryptoxanthin, lutein, luteoxanthin, lycopin and zeaxanthin, chamomilla recutita (matricaria) flower extract, sesamin, pyrus malus fruit extract, baicalein, puerarin, phlorizin, 1,4-dicaffeoylquinic acid, myricetin-3-o-p-rhamnoside, dihydromyricetin, diosmetin, 6-gingerol and mangnolol.

Preferred cosmetic oils comprised in the composition according to the instant invention are selected from the group comprising, preferably consisting of, argan oil, almond oil, blackcurrant oil, chia oil, Cannabis sativa I. oil, avocado oil (persea gratissima), caprylic/capric triglycerides, cotton seed oil, hemp oil, canola oil, marula oil, peach kernel oil, perilla oil, pomegranate seed oil, pumpkin seed oil, sea buckthorn oil, coconut oil, coconut oil-derived fatty acids, glycine soja oil, Helianthus annuus seed oil, jojoba oil, macadamia nut oil, persea gratissima oil, Triticum vulgare (wheat) germ oil, grape seed oil, MCT oil, grapefruit oil, ginger oil, butyrospermum parkii (shea) butter, Camellia sinensis seed oil, cocoa butter, coconut oil and evening primrose oil.

It is an advantage of the instant invention, that a multitude of different cosmetic, pharmaceutical and/or nutraceutical active ingredients can be comprised in the composition according to the instant invention.

Preferably the composition according to the instant invention comprises as component E) at least one ceramide and cholesterol, more preferably at least one ceramide, at least one sphingoid base and cholesterol.

The weight ratio of the sum of all ceramides and as may be the case all sphingoid bases comprised in the composition according to the instant invention to cholesterol in this context is preferably in the range of from 1000:1 to 1:100, more preferably from 100:1 to 1:10 and most preferably from 10:1 to 1:1, respectively.

The compositions according to the instant invention are liposomal compositions, thus they comprise liposomes. Preferably the liposomes comprised in composition according to the instant invention have a mean particle size of 15 nm to 800 nm, preferably of 50 nm to 500 nm, more preferably of 60 nm to 350 nm and even more preferably of 80 nm to 240 nm.

Photon correlation spectroscopy is employed in order to determine the mean particle size. The measurement is performed using a Zetasizer Nano ZS90, Malvern Instruments Ltd., UK, according to the manufacturer's instruction. The Z-average is the intensity weighted mean hydrodynamic size of the ensemble collection of particles measured by dynamic light scattering (DLS). The Z-average is derived from a Cumulants analysis of the measured correlation curve, wherein a single particle size is assumed and a single exponential fit is applied to the autocorrelation function (see Zetasizer Nano ZS90 User Manual MAN0485-1-1 9 Jun. 2017).

A preferred composition according to the instant invention is characterized in that it comprises
A) in an amount of from 0.1 wt.-% to 60 wt.-%, preferably from 1 wt.-% to 40 wt.-%, more preferably from 5 wt.-% to 25 wt.-%,
B) in an amount of from 0.1 wt.-% to 60 wt.-%, preferably from 1 wt.-% to 40 wt.-%, more preferably from 5 wt.-% to 25 wt.-%,
C) in an amount of from 10 wt.-% to 95 wt.-%, preferably from 20 wt.-% to 85 wt.-%, more preferably from 35 wt.-% to 75 wt.-%, as may be the case
D) in an amount of from 0.1 wt.-% to 80 wt.-%, preferably from 1 wt.-% to 60 wt.-%, more preferably from 5 wt.-% to 30 wt.-%, and as may be the case
E) in an amount of from 0.05 wt.-% to 20 wt.-%, preferably from 0.1 wt.-% to 10 wt.-%, more preferably from 0.5 wt.-% to 5 wt.-%, where the percentages by weight refer to the total composition.

This resembles a concentrated stock of a liposomal composition.

In case component E) is comprised, this preferred composition according to the instant invention is very well suited as a storage vehicle for cosmetic, pharmaceutical and/or nutraceutical active ingredients.

These compositions allow for easy handling of the actives and also for formulating end customer formulations with ease.

Thus, the present invention further relates to formulations, especially cosmetic, pharmaceutical, dermatological or nutraceutical formulations, most preferably cosmetic formulations, comprising the composition according to the instant invention.

The formulations according to the invention can further comprise at least one additional component selected from the group of
emollients,
emulsifiers,
thickeners/viscosity regulators/stabilizers,
UV light protection filters,
antioxidants,
hydrotropes,
solids and fillers,
film formers,
pearlescence additives,
deodorant and antiperspirant active ingredients,
insect repellents,
self-tanning agents,
preservatives,
conditioning agents,
perfumes,
dyes,
odour absorbers,
superfatting agents,
other solvents.

Substances which can be used as exemplary representatives of the individual groups are known to those skilled in the art and can be found for example in German application DE 102008001788.4.

This patent application is hereby incorporated as reference and thus forms part of the disclosure.

As regards further optional components and the amounts used of these components, reference is made expressly to the relevant handbooks known to those skilled in the art, for example K.

Schrader, “Grundlagen und Rezepturen der Kosmetika [Cosmetics—fundamentals and formulations]”, 2nd edition, pages 329 to 341, Huthig Buch Verlag Heidelberg.

The amounts of the particular additives are determined by the intended use.

Typical boundary formulations for the respective applications are known prior art and are contained for example in the brochures of the manufacturers of the particular base and active ingredients.

These existing formulations can generally be adopted unchanged. However, if required, for adjustment and optimization, the desired modifications can be undertaken by simple tests without complication.

Preferred formulations according to the instant invention are characterized in that they comprise
A) in an amount of from 0.0001 wt.-% to 6 wt.-%, preferably from 0.001 wt.-% to 4 wt.-%, more preferably from 0.005 wt.-% to 2.5 wt.-%,
B) in an amount of from 0.0001 wt.-% to 6 wt.-%, preferably from 0.001 wt.-% to 4 wt.-%, more preferably from 0.005 wt.-% to 2.5 wt.-%,
C) in an amount of from 20 wt.-% to 99 wt.-%, preferably from 30 wt.-% to 95 wt.-%, more preferably from 50 wt.-% to 90 wt.-%, as may be the case
D) in an amount of from 0.0001 wt.-% to 30 wt.-%, preferably from 0.001 wt.-% to 25 wt.-%, more preferably from 0.005 wt.-% to 20 wt.-%, and as may be the case
E) in an amount of from 0.00005 wt.-% to 40 wt.-%, preferably from 0.0001 wt.-% to 20 wt.-%, more preferably from 0.0005 wt.-% to 0.5 wt.-%, where the percentages by weight refer to the total formulation and preferably with the weight ratio of component C) to component A) being bigger than 10, preferably bigger than 30.

The components A) to E) are the same as comprised in the composition according to the instant invention. D) and E) can be comprised in higher ratios compared to the other components than in the composition according to the instant invention. For example, it is common to add further solvent (component D)) or cosmetic oils (component E)) to end customer formulations.

A particularly preferred composition according to the invention and also a preferred formulation according to the instant invention is characterized in that it has a pH in the range of 4.0 to 8.0, preferably 4.5 to 7.5 particularly preferably 4.5 to 6.5.

The “pH” in connection with the present invention is defined as the value which is measured for the relevant composition at 22° C. after stirring for five minutes using a pH electrode calibrated in accordance with ISO 4316 (1977).

The present invention further relates to a process for encapsulating at least one cosmetic, pharmaceutical and/or nutraceutical active ingredient comprising the steps
I) providing at least one phospholipid A), at least one biosurfactant B), the at least one cosmetic, pharmaceutical and/or nutraceutical active ingredient E) and optionally at least one solvent selected from the group of alcohols comprising at least three carbon atoms and polyols D), and combining the above,
II) heating the provided components to a temperature in the range of from 50° C. to 100° C., preferably from 60° C. to 95° C., more preferably from 70° C. to 90° C., even more preferably from 75° C. to 90° C., and
III) heating water C) to a temperature in the range of from 50° C. to 100° C., preferably from 60° C. to 95° C., more preferably from 70° C. to 90° C., even more preferably from 75° C. to 90° C. and combining the water with the components provided in step I).

Preferably the same preferred components A) to E) are used in the same preferred embodiments in the process of the instant invention as described above for the composition according to the instant invention.

The combining of the components A) to E) in process step III) of the process of the instant invention is preferably done while stirring, preferably while homogenizing by stirring.

Preferably an additional homogenization is carried out. This comprises for example a high shear rate homogenization with a stirrer speed of from 3,000 rpm to 25,000 rpm and typically at 5,000 rpm to 20,000 rpm and/or high pressure homogenization, preferably at from 100 bar to 1000 bar and typically at 500 bar to 700 bar.

The present invention further relates to the use of at least on composition according to the instant invention for encapsulation of at least one cosmetic, pharmaceutical and/or nutraceutical active ingredient. The active ingredient to be encapsulated is preferably those of component E) of the composition according to the instant invention.

The examples adduced hereinafter describe the present invention by way of example, without any intention that the invention, the scope of application of which is apparent from the entirety of the description and the claims, be restricted to the embodiments specified in the examples.

### EXAMPLES

Rhamnolipids were prepared as described in EP3023431.

The sophorolipid used is a sophorolipid REWOFERM SL ONE from Evonik, which has a lactone to acid ratio of 40:60.

Glucolipids were produced according to example 2 of WO2019154970 via fermentation.

### Example 1: Preparation of Liposomes Containing Rhamnolipids (According to the Invention)

In order to produce liposomal composition according to example 1, the lecithin was stirred together with the glycerol and rhamnolipid at a temperature of 85° C. for 60 minutes.

The water, which had also been pre-heated to 85° C., was incorporated into the composition above while homogenizing for 4 minutes. The preservative Optiphen BD was finally added with homogenizing for another 1 minute.

The final liposomal composition had the following content; 100 grams in total were prepared:
11.5 wt.-% lecithin from sunflowers,
22.0 wt.-% glycerol,
11.5 wt.-% rhamnolipid,
54.0 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) were checked using photon correlation spectroscopy (PCS, Zetasizer Nano ZS90, Malvern Instruments Ltd., UK) according to the manufacturer's instruction; the production process was terminated after a mean particle size with a diameter of about 190 nm and PDI<0.2.

### Example 2: Preparation of Liposomes Containing Rhamnolipids and Loaded with Ceramide NP, phytosphingosine and cholesterol as active ingredients (according to the invention)

In order to produce liposomal composition according to example 2, the lecithin was stirred together with the glycerol and rhamnolipid at a temperature of 85° C. for 60 minutes. The active ingredients were added and stirring was conducted for additional 60 minutes.

The water, which had also been pre-heated to 85° C., was incorporated into the composition above while homogenizing for 4 minutes. The preservative Optiphen BD was finally added with homogenizing for another 1 minute.

The final liposomal composition had the following content; 100 grams in total were prepared:
11.5 wt.-% lecithin from sunflowers,
22.0 wt.-% glycerol,
11.5 wt.-% rhamnolipid,
1.2 wt.-% ceramide NP,
1.2 wt.-% phytosphingosine,
0.2 wt.-% cholesterol,
51.4 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) were checked using photon correlation spectroscopy (PCS, Zetasizer Nano ZS90, Malvern Instruments Ltd., UK) according to the manufacturer's instruction; the production process was terminated after a mean particle size with a diameter of about 160 nm and PDI<0.2.

### Example 3: Preparation of Liposomes Containing Rhamnolipids and Loaded with Ceramide NP, Phytosphingosine and Cholesterol as Active Ingredients (According to the Invention)

In order to produce liposomal composition according to example 3, the lecithin is stirred together with the glycerol, 1,2-pentanediol and 1,3-butanediol and rhamnolipid at a temperature of 85° C. for 60 minutes. The active ingredient is added and stirring is conducted for additional 60 minutes.

The water, which has also been pre-heated to 85° C., is incorporated into the composition above while homogenizing for 4 minutes.

The final liposomal composition has the following content; 100 grams in total are prepared:
11.5 wt.-% lecithin from sunflowers,
14 wt.-% glycerol,
2.0 wt.-% 1,2-pentanediol,
3.5 wt.-% 1,3-butanediol
11.5 wt.-% rhamnolipid,
1.2 wt.-% ceramide NP,
1.2 wt.-% phytosphingosine,
0.2 wt.-% cholesterol,
54.9 wt.-% water

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and a PDI of 0.2.

### Example 4: Preparation of Liposomes Containing Polysorbate-80 (not According to the Invention)

In order to produce liposomal composition according to example 4, the lecithin was stirred together 30 with the glycerol and polysorbate-80 at a temperature of 85° C. for 60 minutes.

The water, which had also been pre-heated to 85° C., was incorporated into the composition above while homogenizing for 4 minutes. The preservative Optiphen BD was finally added with homogenizing for another 1 minute.

The final liposomal composition had the following content; 100 grams in total were prepared:
11.5 wt.-% lecithin from sunflowers,
22 wt.-% glycerol,
11.5 wt.-% polysorbate-80,
54 wt.-% water,
1 wt.-% preservative Optiphen BD (Benzyl Alcohol, Benzoic Acid, Dehydroacetic Acid)

The particle size of the lipid vesicles produced by this process and the polydispersity index (PDI) were checked using photon correlation spectroscopy (PCS, Zetasizer Nano ZS90, Malvern Instruments Ltd., UK) according to the manufacturer's instruction; the production process was terminated after a mean particle size with a diameter of about 106 nm and PDI<0.2.

### Example 5: Preparation of Liposomes Containing Polysorbate-80 and Loaded with Ceramide NP, Phytosphingosine and Cholesterol as Active Ingredients (not According to the Invention)

In order to produce liposomal composition according to example 5, the lecithin was stirred together with the glycerol and polysorbate-80 at a temperature of 85° C. for 60 minutes. The active ingredients were added and stirring was conducted for additional 60 minutes.

The water, which had also been pre-heated to 85° C., was incorporated into the composition above while homogenizing for 4 minutes. The preservative Optiphen BD was finally added with homogenizing for another 1 minute.

The final liposomal composition had the following content; 100 grams in total were prepared:
11.5 wt.-% lecithin from sunflowers,
22 wt.-% glycerol,
11.5 wt.-% polysorbate-80,
1.2 wt.-% ceramide NP,
1.2 wt.-% phytosphingosine,
0.2 wt.-% cholesterol,
51.4 wt.-% water,
1 wt.-% preservative Optiphen BD (Benzyl Alcohol, Benzoic Acid, Dehydroacetic Acid)

The particle size of the lipid vesicles produced by this process and the polydispersity index (PDI) were checked using photon correlation spectroscopy (PCS, Zetasizer Nano ZS90, Malvern Instruments Ltd., UK) according to the manufacturer's instruction; the production process was terminated after a mean particle size with a diameter of about 160 nm and PDI<0.3.

### Example 5b: Preparation of Liposomes without Surfactant and Loaded with Ceramide NP, Phytosphingosine and Cholesterol as Active Ingredients (not According to the Invention)

In order to produce liposomal composition according to example 5b, the lecithin was stirred together with the glycerol at a temperature of 85° C. for 60 minutes. The active ingredient was added and stirring was conducted for additional 60 minutes.

The water, which had also been pre-heated to 85° C., was incorporated into the composition above while homogenizing for 4 minutes. The preservative Optiphen BD was finally added with homogenizing for another 1 minute.

The final liposomal composition had the following content; 100 grams in total were prepared:
11.5 wt.-% lecithin from sunflowers,
22 wt.-% glycerol,
1.2 wt.-% ceramide NP,
1.2 wt.-% phytosphingosine,
0.2 wt.-% cholesterol,
62.9 wt.-% water,
1 wt.-% preservative Optiphen BD (Benzyl Alcohol, Benzoic Acid, Dehydroacetic Acid)

No stable liposomal compositions could be prepared with the above components, no particle size or PDI could thus be determined.

### Example 6: Preparation of Liposomes Containing Rhamnolipids and Loaded with Caprooyl Phytosphingosine as Active Ingredient (According to the Invention)

In order to produce liposomal composition according to example 11, the lecithin was stirred together with the glycerol and rhamnolipid at a temperature of 85° C. for 60 minutes. The active ingredient was added and stirring was conducted for additional 60 minutes.

The water, which had also been pre-heated to 85° C., was incorporated into the composition above while homogenizing for 4 minutes. The preservative Optiphen BD was finally added with homogenizing for another 1 minute.

The final liposomal composition had the following content; 100 grams in total were prepared:
8.7 wt.-% lecithin from sunflowers,
18.0 wt.-% glycerol,
8.7 wt.-% rhamnolipid,
3.2 wt.-% caprooyl phytosphingosine,
60.4 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) were checked using photon correlation spectroscopy (PCS, Zetasizer Nano ZS90, Malvern Instruments Ltd., UK) according to the manufacturer's instruction; the production process was terminated after a mean particle size with a diameter of about 218 nm and PDI<0.3.

### Example 7: Preparation of Liposomes Containing Polysorbate-80 and Loaded with Caprooyl Phytosphingosine as Active Ingredient (not According to the Invention)

In order to produce liposomal composition according to example 12, the lecithin was stirred together with the glycerol and polysorbate-80 at a temperature of 85° C. for 60 minutes. The active ingredient was added and stirring was conducted for additional 60 minutes.

The water, which had also been pre-heated to 85° C., was incorporated into the composition above while homogenizing for another 4 minutes. The preservative Optiphen BD was finally added with homogenizing for another 1 minute.

The final liposomal composition had the following content; 100 grams in total were prepared:
8.7 wt.-% lecithin from sunflowers,
18.0 wt.-% glycerol,
8.7 wt.-% polysorbate-80,
3.2 wt.-% caprooyl phytosphingosine,
60.4 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) were checked using photon correlation spectroscopy (PCS, Zetasizer Nano ZS90, Malvern Instruments Ltd., UK) according to the manufacturer's instruction; the production process was terminated after a mean particle size with a diameter of about 200 nm and a PDI 0.5.

In comparison to example 6 it proven by the high PDI, that the liposomal composition of example 7 is not properly stable.

### Example 8: Diminished Crystallisation of Actives Due to Biosurfactant in the Liposomes During Storage

The compositions of examples 2, 3 and 5 were stored at different temperatures and examined for occurrence of crystals at different time point: Crystallization is observable two weeks after storage at room temperature and 4000 for example 5, whereas example 2 and 3 show no crystallisation at all.

### Example 8b: Diminished Separation of Actives Due to Biosurfactant in the Liposomes During Storage

The following liposomal compositions are prepared according to example H2 of EP1 138311:

| Preparation | V1 | 8Rr | 8Rs | 8Rg | V2 | 8RR | 8RS | 8RG |
|---|---|---|---|---|---|---|---|---|
| Squalane | 15 | 15 | 15 | 15 | | | | |
| Dicaprylyl Carbonate | | | | | 15 | 15 | 15 | 15 |
| Polyglyceryl-2 | 5 | 5 | 5 | 5 | 5 | 5 | 5 | 5 |
| Dipolyhydroxystearate | | | | | | | | |
| salicylic acid 2-ethylhexyl | 0.635 | 0.635 | 0.635 | 0.635 | 0.635 | 0.635 | 0.635 | 0.635 |
| ester | | | | | | | | |
| soya lecithin | 1.269 | 1.269 | 1.269 | 1.269 | 1.269 | 1.269 | 1.269 | 1.269 |
| propylene glycol | 0.952 | 0.952 | 0.952 | 0.952 | 0.952 | 0.952 | 0.952 | 0.952 |
| Coco Glucosides | 0.144 | | | | 0.144 | | | |
| rhamnolipids | | 0.144 | | | | 0.144 | | |
| sophorolipids | | | 0.144 | | | | 0.144 | |
| glucolipids | | | | 0.144 | | | | 0.144 |
| Water | ad | ad | ad | ad | ad | ad | ad | ad |
| | 100 | 100 | 100 | 100 | 100 | 100 | 100 | 100 |

Separation of salicylic acid 2-ethylhexyl ester is observable 28 weeks after storage at room temperature and 12 weeks after storage at 4000 for preparation V1 and V2, whereas 8Rr, 8Rs, 8Rg, 8RR, 8RS, 8RG show no separation at all.

### Example 8c: Diminished Separation of Actives Due to Biosurfactantin the Liposomes During Storage

The following liposomal compositions are prepared according to example H2 of EPi1138311 without Squalane:

| Preparation | V3 | 9Rr | 9Rs | 9Rg |
|---|---|---|---|---|
| Polyglyceryl-2 | 5 | 5 | 5 | 5 |
| Dipolyhydroxystearate | | | | |
| Salicylic acid 2-ethylhexyl ester | 0.635 | 0.635 | 0.635 | 0.635 |
| Soya lecithin | 1.269 | 1.269 | 1.269 | 1.269 |
| Propylene glycol | 0.952 | 0.952 | 0.952 | 0.952 |
| Coco Glucosides | 0.144 | | | |
| Rhamnolipids | | 0.144 | | |
| Sophorolipids | | | 0.144 | |
| Glucolipids | | | | 0.144 |
| Water | ad 100 | ad 100 | ad 100 | ad 100 |

Separation of salicylic acid 2-ethylhexyl ester is observable 28 weeks after storage at room temperature and 9 weeks after storage at 40° C. for preparation V3, whereas 9Rr, 9Rs and 9Rg show no separation at all.

### Example 9: Preparation of Liposomes Containing Glucolipids and Loaded with Ceramide NP, Phytosphingosine and Cholesterol as Active Ingredient (According to the Invention)

In order to produce liposomal composition according to example 9, the lecithin is stirred together with the glycerol and glucolipid at a temperature of 85° C. for 60 minutes. The active ingredients are added and stirring is conducted for additional 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared: Glucolipids were produced as described in the examples of WO2019154970.
11.5 wt.-% lecithin from sunflowers,
22.0 wt.-% glycerol,
11.5 wt.-% glucolipid,
1.2 wt.-% ceramide NP,
1.2 wt.-% phytosphingosine,
0.2 wt.-% cholesterol,
51.4 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 10: Preparation of Liposomes Containing Sophorolipids and Loaded with Ceramide NP, Phytosphingosine and Cholesterol as Active Ingredient (According to the Invention)

In order to produce liposomal composition according to example 10, the lecithin is stirred together with the glycerol and sophorolipid at a temperature of 85° C. for 60 minutes. The active ingredients are added and stirring is conducted for additional 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared: The sophorolipid used is a sophorolipid “SL 18” from Ecover, which has an acid to lactone ratio of 70:30.
11.5 wt.-% lecithin from sunflowers,
22 wt.-% glycerol,
11.5 wt.-% sophorolipids,
1.2 wt.-% ceramide NP,
1.2 wt.-% phytosphingosine,
0.2 wt.-% cholesterol,
51.4 wt.-% water,
1 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 11: Preparation of Liposomes Containing PEG-6 Caprylic/Capric Glycerides and Loaded with Ceramide NP, Phytosphingosine and Cholesterol as Active Ingredient (not According to the Invention)

The final liposomal composition has the following content; 100 grams in total are prepared:
11.5 wt.-% lecithin from sunflowers,
22 wt.-% glycerol,
11.5 wt.-% PEG-6 Caprylic/Capric Glycerides,
1.2 wt.-% ceramide NP,
1.2 wt.-% phytosphingosine,
0.2 wt.-% cholesterol,
51.4 wt.-% water,
1 wt.-% preservative Optiphen BD (Benzyl Alcohol, Benzoic Acid, Dehydroacetic Acid)

In order to produce liposomal composition according to example 9, the lecithin is stirred together with the glycerol and PEG-6 Caprylic/Capric Glycerides at a temperature of 85° C. for 60 minutes. The active ingredients are added and stirring was conducted for additional 60 minutes. The water, which has also been pre-heated to 85° C., is incorporated into the composition above while homogenizing for 4 minutes. The preservative Optiphen BD is finally added with stirring for another 1 minute.

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 12: Diminished Re-Crystallisation Due to Biosurfactant in the Liposomes in Repeated Freeze/Thaw Cycles

The liposomal compositions of example 9 to 11 are deep frozen in a −20° C. refrigerator for 3 hours and thawed in a 30° C. water bath. This procedure is repeated four times and the compositions are subsequently analyzed for crystal occurrence.

Crystallization is observed after repeated freeze/thaw cycles in example 11, whereas example 9 and 10 show no re-crystallisation at all.

### Example 13: Enhanced Liposome Stability Due to Biosurfactant in the Liposomal Composition

The physical stability of the formulations and the resulting liposome stability is determined by pH value, particle size and polydispersity index measurements using a pH meter and photon correlation spectroscopy (PCS, Zetasizer Nano ZS90, Malvern Instruments Ltd., UK) according to the manufacturer's instruction.

Formulations are stored at 4° C., room temperature and 40° C. and measured over the course of 48 weeks.

Strongly affected stability of the liposomes is given, when the pH shifts more than +/−2 units, liposome particle size increases by more than 400 nm and/or the polydispersity index is above 0.4.

### Example 14: Preparation of Liposomes Containing Rhamnolipids (According to the Invention)

In order to produce liposomal composition according to example 14, the lecithin is stirred together with the glycerol and rhamnolipid at a temperature of 85° C. for 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared:
5.5 wt.-% lecithin from sunflowers,
25.0 wt.-% glycerol,
4.5 wt.-% rhamnolipid,
64 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 15: Preparation of Liposomes Containing Glucolipids (According to the Invention)

In order to produce liposomal composition according to example 15, the lecithin is stirred together with the glycerol and glucolipid at a temperature of 85° C. for 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared:
5.5 wt.-% lecithin from sunflowers,
25.0 wt.-% glycerol,
4.5 wt.-% glucolipid,
64 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 16: Preparation of Liposomes Containing Sophorolipids (According to the Invention)

In order to produce liposomal composition according to example 16, the lecithin is stirred together with the glycerol and sophorolipid at a temperature of 85° C. for 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared:
5.5 wt.-% lecithin from sunflowers,
25.0 wt.-% glycerol,
4.5 wt.-% sophorolipid,
64 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 17: Preparation of Liposomes Containing Rhamnolipids and Loaded with Sphinganine as Active Ingredient (According to the Invention)

In order to produce liposomal composition according to example 17, the lecithin is stirred together with the glycerol and rhamnolipid at a temperature of 85° C. for 60 minutes. The active ingredient is added and stirring is conducted for additional 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared:
5.5 wt.-% lecithin from sunflowers,
25.0 wt.-% glycerol,
4.5 wt.-% rhamnolipid,
1.5 wt.-% sphinganine,
62.5 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 18: Preparation of Liposomes Containing Glucolipids and Loaded with Sphinganine as Active Ingredient (According to the Invention)

In order to produce liposomal composition according to example 18, the lecithin is stirred together with the glycerol and glucolipid at a temperature of 85° C. for 60 minutes. The active ingredient is added and stirring is conducted for additional 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared:
5.5 wt.-% lecithin from sunflowers,
25.0 wt.-% glycerol,
4.5 wt.-% glucolipid,
1.5 wt.-% sphinganine,
62.5 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 19: Preparation of Liposomes Containing Sophorolipids and Loaded with Sphinganine as Active Ingredient (According to the Invention)

In order to produce liposomal composition according to example 19, the lecithin is stirred together with the glycerol and sophorolipid at a temperature of 85° C. for 60 minutes. The active ingredient is added and stirring is conducted for additional 60 minutes.

The water, which has also been pre-heated to 85° C., is incorporated into the composition above while homogenizing for 4 minutes. The preservative Optiphen BD is finally added with homogenizing for another 1 minute.

The final liposomal composition has the following content; 100 grams in total are prepared:
5.5 wt.-% lecithin from sunflowers,
25.0 wt.-% glycerol,
4.5 wt.-% sophorolipid,
1.5 wt.-% sphinganine,
62.5 wt.-% water,
1.0 wt.-% preservative Optiphen BD (Benzyl Alcohol, Benzoic Acid, Dehydroacetic Acid)

The particle size of the lipid vesicles produced by this process and the polydispersity index (PDI) are checked using photon correlation spectroscopy (PCS, Zetasizer Nano ZS90, Malvern Instruments Ltd., UK) according to the manufacturer's instruction; the production process is terminated after a mean particle size with a diameter of about 200 nm and PDI<0.2.

### Example 20: Formulation Examples are Listed Containing Preparations of Liposomes of the Examples

2, 3, 6, 9, 10, 17, 18 or 19; thus, for each formulation listed 8 formulations are prepared. “example X”=examples 2, 3, 6, 9, 10, 17, 18 or 19.

### 20.1 Cream

A 1.50 wt.-% TEGO® Care 450 MB (Polyglyceryl-3 Methylglucose Distearate)
   0.50 wt.-% ABIL® Care XL 80 MB (Bis-PEG/PPG-20/5 PEG/PPG-20/5 Dimethicone;
   Methoxy PEG/PPG-25/4 Dimethicone; Caprylic/Capric Triglyceride)
   1.50 wt.-% Stearyl Alcohol
   2.50 wt.-% TEGIN® M Pellets MB (Glyceryl Stearate)
   6.50 wt.-% TEGOSOFT® M (Isopropyl Myristate)
   4.80 wt.-% TEGOSOFT® APM (PPG-3 Myristyl Ether)
   0.50 wt.-% TEGOSOFT® MM MB (Myristyl Myristate)
B 1.00 wt.-% TEGO® Natural Betaine (Betaine)
   3.00 wt.-% Glycerin
   ad 100 wt.-% Water
C 0.20 wt.-% Carbomer
   0.10 wt.-% Xanthan Gum
   0.80 wt.-% TEGOSOFT® M (Isopropyl Myristate)
D 3.00 wt.-% example X (see above)
   q.s. Sodium Hydroxide (10% in water)
   1.00 wt.-% Verstatil® PC (Phenoxyethanol; Caprylyl Glycol)
   q.s. Perfume

### 20.2 Facial Mask

A 0.50 wt.-% ABIL® Care XL 80 MB (Bis-PEG/PPG-20/5 PEG/PPG-20/5 Dimethicone;
   Methoxy PEG/PPG-25/4 Dimethicone; Caprylic/Capric Triglyceride)
   1.50 wt.-% ABIL® 350 (Dimethicone)
   4.00 wt.-% TEGOSOFT® OS (Ethylhexyl Stearate)
   4.00 wt.-% TEGOSOFT® CT MB (Caprylic/Capric Triglyceride)
B 0.60 wt.-% TEGO® Carbomer 341 ER (Acrylates/C10-30 Alkyl Acrylate Crosspolymer)
   0.10 wt.-% HyaCare® (Sodium Hyaluronate)
   0.10 wt.-% HyaCare® 50 (Hydrolyzed Hyaluronic Acid)
   5.00 wt.-% Glycerin
   ad 100 wt.-% Water
C 3.00 wt.-% example X (see above)
D 1.00 wt.-% Verstatil® PC (Phenoxyethanol; Caprylyl Glycol)
E q.s. Sodium Hydroxide (10% in water)

### 20.3 Serum

A 0.30 wt.-% TEGO® Carbomer 341 ER (Acrylates/C10-30 Alkyl Acrylate Crosspolymer)
   ad 100 wt.-% Water
B q.s. Sodium Hydroxide (10% in water)
C 1.00 wt.-% TEGO® SML 20 MB (Polysorbate 20)
   3.00 wt.-% example X (see above)
D 2.00 wt.-% Verstatil® PC (Phenoxyethanol; Caprylyl Glycol)

### 20.4 Cream

A ad 100 wt.-% Water, deionized
   2.00 wt.-% Glycerin 99,5%, Ph. Eur.(Glycerin)
   0.10 wt.-% dermofeel® PA-3 (Sodium Phytate; Aqua; Alcohol)
A1 0.30 wt.-% Xanthan Gum FEDCS-PC (Xanthan Gum)
B 1.00 wt.-% TEGO® Feel C 10 (Cellulose)
   6.00 wt-% symbio® muls GC MB (Glyceryl Stearate Citrate; Cetearyl Alcohol; Glyceryl Caprylate)
   6.00 wt.-% TEGOSOFT® DC MB (Decyl Cocoate)
   2.00 wt.-% dermosoft® decalact sebum MB (Polyglyceryl-10 Laurate; Sodium Caproyl/Lauroyl Lactylate; Glycerin, Serenoa Serrulata Fruit Extract; Juniperus Communis Fruit Extract)
   0.10 wt.-% dermofeel® Toco 70 non GMO (Tocopherol; Helianthus Annuus (Sunflower) Seed Oil)
C 3.00 wt.-% dermosoft® 1388 eco (Glycerin; Aqua; Sodium Levulinate; Sodium Anisate)
   3.00 wt.-% example X (see above)
   0.15 wt.-% Perfume

### 20.5 Lip Mask

A 0.02 wt.-% HyaCare® (Sodium Hyaluronate)
   9.96 wt.-% Glycerin
   ad 100 wt.-% Water
   0.02 wt.-% Rouge Covarine W 3799 (C112490;Aqua;Glycerin)
   0.02 wt.-% Rouge Covarine W 3799 (C112490;Aqua;Glycerin)
B 8.00 wt.-% TEGOSOFT® DC MB (Decyl Cocoate)
   2.00 wt.-% TEGOSOFT® OER MB (Oleyl Erucate)
   3.00 wt.-% TEGOSOFT® MM MB (Myristyl Myristate)
   7.00 wt.-% TEGOSOFT® P (Isopropyl Palmitate)
   3.00 wt.-% ISOLAN® GPS (Polyglyceryl-4 Diisostearate/Polyhydroxystearate/Sebacate)
   6.00 wt.-% TEGOSOFT® SH MB (Stearyl Heptanoate)
   1.00 wt.-% Cera Alba
   10.00 wt.-% Candelilla Wax (Candelilla Cera)
   3.00 wt.-% Copernicia Cerifera (Carnauba) Wax)
   3.50 wt.-% Butyrospermum Parkii (Shea) Butter)
   33.49 wt.-% SAT-Y-338073 (Yellow Iron Oxide)
   33.49 wt.-% Schercemol PTID Ester (Triisostearoyl Polyglyceryl-3 Dimer Dilinoleate)
C 2.00 wt.-% example X (see above)

### 20.6. Sleep Mask

A 0.50 wt.-% ABIL® Care XL 80 MB (Bis-PEG/PPG-20/5 PEG/PPG-20/5 Dimethicone;
   Methoxy PEG/PPG-25/4 Dimethicone; Caprylic/Capric Triglyceride)
   1.50 wt.-% ABIL® Wax 9840 (Cetyl Dimethicone)
   4.00 wt.-% TEGOSOFT® OS (Ethylhexyl Stearate)
   4.00 wt.-% TEGOSOFT® CT MB (Caprylic/Capric Triglyceride)
B ad 100 wt.-% Water
   5.00 wt.-% Glycerin
   0.05 wt.-% KELCOGEL CG-HA (Gellan Gum)
   0.40 wt.-% Carbomer (Carbomer Homopolymer C)
C 2.00 wt.-% TEGO® Natural Betaine (Betaine)
   0.50 wt.-% TEGO® Turmerone (Curcuma longa (turmeric) root extract)
   2.00 wt.-% example X (see above)
D q.s. Sodium Hydroxide (10% in water)
E 0.80 wt.-% Verstatil® PC (Phenoxyethanol; Caprylyl Glycol)

### 20.7 Body Mask

A ad 100 wt.-% Water, deionized
   4.50 wt.-% Glycerin 99,5%, Ph. Eur. (Glycerin)
   0.10 wt.-% dermofeel® PA-3 (Sodium Phytate; Aqua; Alcohol)
   0.80 wt.-% Xanthan Gum FEDCS-PC (Xanthan Gum)
B 5.00 wt.-% Phytosqualan, refined, Olive based (Squalane)
B1 8.00 wt.-% symbio® muls GC MB (Glyceryl Stearate Citrate; Cetearyl Alcohol; Glyceryl Caprylate)
   2.00 wt.-% TEGIN® M Pellets MB (Glyceryl Stearate)
   2.00 wt.-% KahlWax 8104 (Cera Alba)
   2.00 wt.-% KahlWax 6614 (Camellia Sinensis Leaf Extract)
   2.00 wt.-% Sheabutter, refined, organic (Butyrospermum Parkii Butter)
   5.00 wt.-% Almond Oil, refined (Prunus Amygdalus Dulcis Oil)
   3.00 wt.-% Avocado Oil, refined (Persea Gratissima Oil)
   2.00 wt.-% Apricot Kernel Oil, refined (Prunus Armeniaca Kernel Oil)
   0.20 wt.-% dermofeel® Toco 70 non GMO (Tocopherol; Helianthus Annuus (Sunflower) Seed Oil)
   1.00 wt.-% dermofeel® TocoSkin (Tocopherol; Helianthus Annuus (Sunflower) Seed Oil)
C 0.50 wt.-% TEGO® Turmerone (Curcuma longa (turmeric) root extract)
   0.50 wt.-% Pumpkin seed (Kurbiskern) organic extractive P-00025487 (Helianthus Annuus Hybrid Oil; Cucurbita Pepo Seed Extract; Rosmarinus Officinalis Leaf Extract)
   2.00 wt.-% example X (see above)
   3.5 wt.-% dermosoft® 1388 eco NaL (Glycerin, Aqua, Sodium Levulinate, p-Anisic Acid, Sodium Hydroxide)

### 20.8 After Shave Serum

A ad 100 wt.-% Water, deionized
   0.10 wt.-% dermofeel® PA-3 (Sodium Phytate; Aqua; Alcohol)
   3.00 wt.-% dermosoft® OM (Methylpropanediol; Caprylyl Glycol)
   5.00 wt.-% Glycerin 99,5%, Ph. Eur. (Glycerin)
B 2.50 wt.-% symbio® prot V MB (Hydrolyzed Vegetable Protein; Sodium Citrate; Magnesium Stearate; Xanthan Gum)
   0.50 wt.-% TEGIN® M Pellets MB (Glyceryl Stearate)
   4.00 wt.-% TEGOSOFT® DC MB (Decyl Cocoate)
   0.50 wt.-% Phytosqualan (Squalane)
   1.00 wt.-% TEGO® Feel C 10 (Cellulose)
   0.20 wt.-% dermofeel® Toco 70 non GMO (Tocopherol; Helianthus Annuus (Sunflower) Seed Oil)
   2.00 wt.-% dermosoft® decalact sebum MB (Polyglyceryl-10 Laurate; Sodium Caproyl/Lauroyl Lactylate; Glycerin, Serenoa Serrulata Fruit Extract; Juniperus Communis Fruit Extract)
C 3.00 wt.-% Ethanol denat. (Alcohol Denat.)
D 2.00 wt.-% example X (see above)

### 20.9 Hair & Scalp Serum

A 2.00 wt.-% TEGO® Care LTP MB (Sorbitan Laurate; Polyglyceryl-4 Laurate; Dilauryl Citrate)
   2.67 wt.-% TEGOSOFT® CT MB (Caprylic/Capric Triglyceride)
   1.33 wt.-% dermofeel® sensolv MB (Isoamyl Laurate)
   1.00 wt.-% Hairflux® (Ceramide NG; Olea Europaea (Olive) Fruit Oil; Ricinus Communis (Castor) Seed Oil)
B ad 100 wt.-% Water
   3.00 wt.-% Glycerin
C 2.00 wt.-% dermofeel® sensolv MB (Isoamyl Laurate)
   0.10 wt.-% Carbomer (Carbomer Homopolymer A)
   0.10 wt.-% Acrylates/C10-C30 Alkyl Acrylate Crosspolymer (Carbomer Copolymer C)
D 2.00 wt.-% Water
   0.005 wt.-% TEGO® Pep 3-Boost (Copper Tripeptide 34)
E 2.00 wt.-% example X (see above)
F 1.00 wt.-% Verstatil® PC (Phenoxyethanol; Caprylyl Glycol)
   q.s. Sodium Hydroxide (10% in water)

### 20.10 Low Viscous Face Serum

A 3.00 wt.-% TEGO® Care PBS 6 MB (Polyglyceryl-6 Stearate (and) Polyglyceryl-6 Behenate)
   0.50 wt.-% TEGIN® M Pellets MB (Glyceryl Stearate)
   4.00 wt.-% TEGOSOFT® DEC (Diethylhexyl Carbonate)
   5.00 wt.-% TEGOSOFT® XC MB (Phenoxyethyl Caprylate)
   3.00 wt.-% dermofeel® sensolv MB (Isoamyl Laurate)
   3.00 wt.-% TEGO® SP 13-1 MB (Poly C10-30 Alkyl Acrylate)
B ad 100 wt.-% Water
   3.00 wt.-% Glycerin
   2.00 wt.-% example X (see above)
   0.20 wt.-% Carbomer (Carbomer Homopolymer A)
   0.10 wt.-% HyaCare® 50 (Hydrolyzed Hyaluronic Acid)
C q.s. Sodium Hydroxide (10% in water)
D 1.00 wt.-% Verstatil® PC (Phenoxyethanol; Caprylyl Glycol)
Z q.s. Perfume

### 20.11 Anti-acne Serum

A 2.00 wt.-% TEGOSOFT® AC MB (Isoamyl Cocoate)
   2.00 wt.-% TEGOSOFT® OER MB (Oleyl Erucate)
   1.00 wt.-% Octyldodecanol
   1.00 wt.-% TEGOSOFT® APM (PPG-3 Myristyl Ether)
   0.50 wt.-% TEGO® Turmerone (Curcuma longa (turmeric) root extract)
B 0.50 wt.-% TEGO® Carbomer 341 ER (Acrylates/C10-30 Alkyl Acrylate Crosspolymer)
   5.00 wt.-% Glycerin
   3.00 wt.-% Alcohol
   ad 100 wt.-% Water
C 1.00 wt.-% example X (see above)
   q.s. Sodium Hydroxide
Z q.s. Preservative

### 20.12 O/W After Shave Cream

A 1.5 wt.-% AXOL® C 62 Pellets MB (Glyceryl Stearate Citrate)
   2.0 wt.-% TEGIN® M Pellets MB (Glyceryl Stearate)
   3.0 wt.-% TEGO® Alkanol 1618 MB (Cetearyl Alcohol)
   3.5 wt.-% TEGOSOFT® TN (C12-15 Alkyl Benzoate)
   2.0 wt.-% TEGOSOFT® CR MB (Cetyl Ricinoleate)
   1.0 wt.-% Triisostearin
   0.5 wt.-% TEGOSOFT® MM MB (Myristyl Myristate)
   4.0 wt.-% Cyclopentasiloxane
   2.0 wt.-% Macadamia Ternifolia Seed Oil
   0.5 wt.-% Tocopheryl Acetate
B 4.0 wt.-% Glycerin
   0.5 wt.-% Panthenol
   0.2 wt.-% Allantoin
   ad 100 wt.-% Water
C 0.3 wt.-% Carbomer (Carbomer Homopolymer B)
   1.2 wt.-% TEGOSOFT® TN (C12-15 Alkyl Benzoate)
D 0.5 wt.-% Bisabolol
E q.s. Sodium Hydroxide (10% in water)
F 7.0 wt.-% Alcohol
G 3.0 wt.-% example X (see above)
H 0.7 wt.-% Euxyl PE 9010 (Phenoxyethanol,Ethylhexylglycerin)
Z q.s. Perfume

### 20.13 After-sun gel

A 1.5 wt.-% TEGOSOFT® liquid (Cetearyl Ethylhexanoate)
   0.6 wt.-% ABIL® B 8832 (Bis-PEG/PPG-20/20 Dimethicone)
B 0.3 wt.-% dermosoft® GMCY MB (Glyceryl Caprylate)
   0.5 wt.-% TEGO® Cosmo C 250 (1-Methylhydantoin-2-Imide)
   0.03 wt.-% HyaCare® (Sodium Hyaluronate)
   3.00 wt.-% Glycerin
   3.00 wt.-% Butylene Glycol
   ad 100 wt.-% Water, demineralized
   0.2 wt.-% Carbomer (Carbomer Homopolymer C)
   0.7 wt.-% Aristoflex® HMB (Ammonium Acryloyldimethyltaurate, Behnenth-25 Methacrylate Crosspolymer)
C 1.00 wt.-% dermosoft® Hexiol (1,2-Hexanediol)
   3.00 wt.-% example X (see above)
   q.s. Sodium Hydroxide
   0.25 wt.-% Red 28 (0.02% in water)

### 20.14 Cream SPF 15

A 3.00 wt.-% TEGO® Care PBS 6 MB (Polyglyceryl-6 Stearate (and) Polyglyceryl-6 Behenate)
   8.00 wt.-% TEGOSOFT® XC MB (Phenoxyethyl Caprylate)
   5.00 wt.-% TEGOSOFT® DEC (Diethylhexyl Carbonate)
   3.00 wt.-% TEGIN® M Pellets MB (Glyceryl Stearate)
   2.00 wt.-% TEGO® Alkanol 1618 MB (Cetearyl Alcohol)
   1.00 wt.-% TEGOSOFT® MM MB (Myristyl Myristate)
   0.50 wt.-% ABIL® 350 (Dimethicone)
   0.50 wt.-% TEGO® Feel C 10 (Cellulose)
   2.50 wt.-% Diethylamino Hydroxybenzoyl Hexyl Benzoate
   6.00 wt.-% Homosalate
   0.50 wt.-% Tocopheryl Acetate
B 11.58 wt.-% Water
   0.42 wt.-% Sodium Hydroxide
   3.00 wt.-% Eusolex 232 (Phenylbenzimidazole Sulfonic Acid)
C ad 100 wt.-% Water
   3.00 wt.-% Glycerin
   1.00 wt.-% TEGO® Pep 4-17 MB (Tetrapeptide-21; Glycerin; Butylene Glycol; Aqua)
   0.50 wt.-% TEGO® Cosmo C 100 (Creatine)
D 0.70 wt.-% Keltrol CG-SFT (Xanthan Gum)
E 2.00 wt.-% example X (see above)
   1.10 wt.-% Verstatil® PC (Phenoxyethanol; Caprylyl Glycol)
   0.50 wt.-% Perfume
      20.15 Hand cream SPF 20
A 1.50 wt.-% TEGO® Care PBS 6 MB (Polyglyceryl-6 Stearate (and) Polyglyceryl-6 Behenate)
   2.50 wt.-% VARISOFT® TA 100 (Distearyldimonium Chloride)
   2.00 wt.-% Stearyl Alcohol
   2.00 wt.-% TEGIN® M Pellets MB (Glyceryl Stearate)
   1.00 wt.-% TEGOSOFT® SH MB (Stearyl Heptanoate)
   5.00 wt.-% TEGOSOFT® TN (C12-15 Alkyl Benzoate)
   3.00 wt.-% Diethylamino Hydroxybenzoyl Hexyl Benzoate
   3.00 wt.-% Ethylhexyl Salicylate
   5.00 wt.-% Ethylhexyl Methoxycinnamate
   0.50 wt.-% Tocopheryl Acetate
B ad 100 wt.-% Water
   3.00 wt.-% Glycerin
C 1.50 wt.-% Nylon-12
D 2.00 wt.-% TEGO® Natural Betaine (Betaine)
   2.00 wt.-% Water
E 2.00 wt.-% example X (see above)
   q.s. Preservative
   0.50 wt.-% Perfume “Azura 231517”
      20.16 Sun care lotion SPF 30+ insect repellent
A 3.50 wt.-% TEGO® Care PBS 6 MB (Polyglyceryl-6 Stearate (and) Polyglyceryl-6 Behenate)
   2.00 wt.-% TEGOSOFT® DC MB (Decyl Cocoate)
   1.00 wt.-% TEGOSOFT® TN (C12-15 Alkyl Benzoate)
   0.50 wt.-% REWOPAL® PIB 1000 (Polyisobutene)
   0.50 wt.-% TEGIN® M Pellets MB (Glyceryl Stearate)
   0.50 wt.-% Stearyl Alcohol
   15.00 wt.-% Insect Repellent 3535 (Ethyl Butylacetylaminopropionate)
   3.00 wt.-% Tinosorb S (Bis-Ethylhexyloxyphenol Methoxyphenyl Triazine)
   3.00 wt.-% Ethylhexyl Salicylate
   6.00 wt.-% Homosalate
   8.00 wt.-% Octocrylene
   2.00 wt.-% Butyl Methoxydibenzoylmethane
   2.00 wt.-% Ethylhexyl Methoxycinnamate
B ad 100 wt.-% Water
   3.00 wt.-% Glycerin
C 0.60 wt.-% Sepigel 305 (Polyacrylamide (and) C13-14 Isoparaffin (and) Laureth-7)
D 2.00 wt.-% example X (see above)
   0.70 wt.-% EUXYL PE 9010 (Phenoxyethanol; Ethylhexylglycerin)
Z q.s. Perfume
   20.17 O/W foundation
A 4.00 wt.-% TEGO® Care PBS 6 MB (Polyglyceryl-6 Stearate (and) Polyglyceryl-6 Behenate)
   1.50 wt.-% TEGO® Alkanol 1618 MB (Cetearyl Alcohol)
   1.00 wt.-% TEGIN® M Pellets MB (Glyceryl Stearate)
   1.00 wt.-% TEGOSOFT® MM MB (Myristyl Myristate)
   3.00 wt.-% TEGOSOFT® TN (C12-15 Alkyl Benzoate)
   2.00 wt.-% TEGOSOFT® DEC (Diethylhexyl Carbonate)
   2.00 wt.-% 9045 Silicone Elastomer Blend (Cyclopentasiloxane (and) Dimethicone Crosspolymer)
   8.00 wt.-% Ethylhexyl Methoxycinnamate
   5.00 wt.-% Ethylhexyl Salicylate
   3.00 wt.-% Diethylamino Hydroxybenzoyl Hexyl Benzoate
   3.00 wt.-% Bis-Ethylhexyloxyphenol Methoxyphenyl Triazine
   (Bis-Ethylhexyloxyphenol Methoxyphenyl Triazine)
B ad 100 wt.-% Water, deionized
   1.00 wt.-% Glycerin
   0.15 wt.-% Xanthan Gum
C 8.00 wt.-% Titanium Dioxide; Cl 77891 1.70 wt.-% Covarine Yellow WN 1792 GZ (Cl 77492, Aqua, Glycerin, Xanthan Gum, Sodium Citrate)
   0.40 wt.-% Covarine Red WN 3798 GZ (Cl 77491, Aqua, Glycerin, Xanthan Gum, Sodium Citrate)
   0.10 wt.-% Covarine Black WN 9798 GZ (Cl 77499, Water, Glycerin, Xanthan Gum, Sodium Citrate)
   2.00 wt.-% Glycerin
   3.00 wt.-% Propylene Glycol
D 2.00 wt.-% Nylon-12
E 2.00 wt.-% example X (see above)
   0.70 wt.-% Euxyl PE 9010 (Phenoxyethanol,Ethylhexylglycerin)
   20.18 Face Care Mousse
A 2.50 wt.-% ABIL® Care XL 80 MB (Bis-PEG/PPG-20/5 PEG/PPG-20/5 Dimethicone; Methoxy PEG/PPG-25/4 Dimethicone; Caprylic/Capric Triglyceride)
   5.00 wt.-% Dimethicone (5 mPas)
   4.00 wt.-% TEGOSOFT® DEC (Diethylhexyl Carbonate)
   3.00 wt.-% TEGOSOFT® CT MB (Caprylic/Capric Triglyceride)
   1.00 wt.-% TEGOSOFT® CR MB (Cetyl Ricinoleate)
B 4.00 wt.-% TEGO® Pep UP (Tetrapeptide-4; Glycerin)
   0.50 wt.-% TEGO® Cosmo C 100 (Creatine)
   0.50 wt.-% Panthenol
   0.20 wt.-% Allantoin
   ad 100 wt.-% Water
C 1.60 wt.-% TEGOSOFT® OS (Ethylhexyl Stearate)
   0.15 wt.-% Carbomer (Carbomer Homopolymer A)
   0.15 wt.-% Carbomer (Carbomer Homopolymer C)
   0.10 wt.-% Keltrol CG-SFT (Xanthan Gum)
D q.s. Sodium Hydroxide (10% in water)
E 1.00 wt.-% TEGO® Betain 810 MB (Capryl/Capramidopropyl Betaine)
   1.00 wt.-% REWOTERIC® AM C MB (Sodium Cocoamphoacetate)
   2.00 wt.-% example X (see above)
   0.70 wt.-% Euxyl PE 9010 (Phenoxyethanol,Ethylhexylglycerin)
Z q.s. Perfume

### 20.19 Face Mask

A 1.00 wt.-% TEGOSOFT® PC 41 MB (Polyglyceryl-4 Caprate)
   1.30 wt.-% TEGO® Betain 810 MB (Capryl/Capramidopropyl Betaine)
   1.00 wt.-% TEGO® Solve 61 MB (Polyglyceryl-6 Caprylate; Polyglyceryl-3 Cocoate; Polyglyceryl-4 Caprate; Polyglyceryl-6 Ricinoleate)
   1.00 wt.-% Glycerin
   1.40 wt.-% Hexylene Glycol
B 0.50 wt.-% TEGO® Carbomer 140 (Carbomer)
   2.00 wt.-% TEGO® Natural Betaine (Betaine)
   ad 100 wt.-% Water
   0.10 wt.-% Xanthan Gum
C 2.50 wt.-% TEGO® Feel C 10 (Cellulose)
   0.50 wt.-% TEGO® Turmerone (Curcuma longa (turmeric) root extract)
   3.00 wt.-% Verstatil® BOB (Benzyl Alcohol; Caprylyl Glycol; Benzoic Acid)
   2.00 wt.-% example X (see above)
   q.s. wt.-% Sodium Hydroxide (10% in water)

### 20.20 After Shave Cream

A 2.00 wt.-% AXOL® C 62 Pellets MB (Glyceryl Stearate Citrate)
   2.00 wt.-% TEGIN® M Pellets MB (Glyceryl Stearate)
   3.00 wt.-% TEGO® Alkanol 1618 MB (Cetearyl Alcohol)
   4.00 wt.-% TEGOSOFT® DEC (Diethylhexyl Carbonate)
   3.50 wt.-% TEGOSOFT® DC MB (Decyl Cocoate)
   2.00 wt.-% TEGOSOFT® CR MB (Cetyl Ricinoleate)
   1.00 wt.-% TEGOSOFT® OER MB (Oleyl Erucate)
   0.50 wt.-% TEGOSOFT® MM MB (Myristyl Myristate)
   2.00 wt.-% Macadamia Ternifolia Seed Oil
   0.50 wt.-% Tocopheryl Acetate
   0.50 wt.-% Bisabolol
   0.50 wt.-% dermosoft® GMCY MB (Glyceryl Caprylate)
B ad 100 wt.-% Water
   4.0 wt.-% Glycerin
   0.50 wt.-% Panthenol
   0.20 wt.-% Allantoin
C 0.40 wt.-% Keltrol CG-SFT (Xanthan Gum)
D 7.00 wt.-% Alcohol
E 2.00 wt.-% example X (see above)
   1.50 wt.-% Verstatil® SL non GMO (Aqua; Sodium Levulinate; Potassium Sorbate)

### 20.21 Skin Tonic

A ad 100 wt.-% Water, deionized
   4.00 wt.-% Glycerin 99,5%, Ph. Eur. (Glycerin)
   0.50 wt.-% Ajidew NL-50N (Sodium PCA; Aqua)
   0.10 wt.-% dermofeel® PA-3 (Sodium Phytate; Aqua; Alcohol)
B 1.50 wt.-% symbio® solv clear plus MB (Caprylyl/Capryl Glucoside; Aqua; Sodium Cocoyl Glutamate; Glyceryl Caprylate; Citric Acid; Polyglyceryl-6 Oleate; Sodium Surfactin)
   1.00 wt.-% dermosoft® decalact liquid MB (Sodium Caproyl/Lauroyl Lactylate; Triethyl Citrate)
   25.00 wt.-% Ethanol denat. (Alcohol Denat.)
   0.30 wt.-% Perfume
   2.00 wt.-% dermofeel® TEC eco (Triethyl Citrate)
C 2.00 wt.-% example X (see above)
   20.22 Hair and body shampoo
2.25 wt.-% RHEANCE® One (Glycolipids)
0.50 wt.-% dermofeel® sensolv MB (Isoamyl Laurate)
9.70 wt.-% Lauryl Glucoside, 50%
20.00 wt.-% Coco-Glucoside (50%)
5.50 wt.-% Sodium Coco-Sulfate, 91%
ad 100.00 wt.-% Water
6.80 wt.-% Glycerin
3.50 wt.-% dermosoft® 1388 eco (Glycerin; Aqua; Sodium Levulinate; Sodium Anisate)
1.05 wt.-% TEGO® Remo 95 MB (Sorbitan Caprylate, Glyceryl Oleate)
2.00 wt-% example X (see above)
ad pH with Citric Acid

### 20.23 Micellar Water for Facial Cleansing

2.00 wt.-% TEGO® Solve 55 MB (Polyglyceryl-3 Caprylate/Caprate/Succinate; Propylene Glycol)
1.30 wt.-% TEGO® Betain 810 MB (Capryl/Capramidopropyl Betaine)
0.05 wt.-% Perfume
ad 100.0 wt.-% Water
2.00 wt.-% TEGO® Natural Betaine (Betaine)
1.40 wt.-% Hexylene Glycol
1.00 wt.-% Glycerin
2.00 wt.-% example X (see above)
q.s. Preservative
   20.24 Leave—in Hair conditioner
A 5.00 wt.-% TEGO® Sulfosuccinate DO 75 (Diethylhexyl Sodium Sulfosuccinate)
   10.00 wt.-% dermofeel® sensolv MB (Isoamyl Laurate)
   48.00 wt.-% TEGOSOFT® TN (C12-15 Alkyl Benzoate)
   30.00 wt.-% Helianthus Annuus (Sunflower) Seed Oil)
B 5.00 wt.-% TEGOSOFT® PC 31 MB (Polyglyceryl-3 Caprate)
   1.00 wt.-% VARISOFT® EQ 100 (Bis-(Isostearoyl/Oleoyl Isopropyl) Dimonium Methosulfate)
   1.00 wt.-% example X (see above)

### 20.25 W/O Lotion

A 2.5 wt.-% ISOLAN® GPS (Polyglyceryl-4 Diisostearate/Polyhydroxystearate/Sebacate)
   10.0 wt.-% TEGOSOFT® DEC (Diethylhexyl Carbonate)
   9.0 wt.-% TEGOSOFT® OP (Ethylhexyl Palmitate)
   0.5 wt.-% Zinc Stearate
B ad 100.0 wt.-% Water
   3.0 wt.-% Glycerin
   1.5 wt.-% Magnesium Sulfate Heptahydrate
   2.0 wt.-% example X (see above)
Z q.s. Preservative, Perfume

1. A liposomal composition, comprising:
A) at least one phospholipid, which is at least in part located in the bilayer of the liposomes,
B) at least one biosurfactant,
C) water, and optionally
D) at least one solvent selected from the group consisting of alcohols comprising at least three carbon atoms and polyols.

2. The composition according to claim 1, wherein the at least one phospholipid is at least one selected from the group consisting of phosphatidylcholines, phosphatidylethanolamines, phosphatidic acid, phosphatidylserines, phosphoinositides and phosphosphingolipids.

3. The composition according to claim 1, wherein the at least one biosurfactant is at least one selected from the group consisting of rhamnolipids, sophorolipids and glucolipids.

4. The composition according to claim 1 wherein the at least one solvent is an alcohol which is at least one selected from the group consisting of alkanols and alkenols.

5. The composition according to claim 1, wherein the at least one solvent is an alcohol which is at least one selected from the group consisting of diols and triols.

6. The composition according to claim 1 further comprising:
E) at least one cosmetic, pharmaceutical and/or nutraceutical active ingredient with a water solubility at 25° C. and 1 bar of less than 1 mg/ml.

7. The composition according to claim 6, wherein the at least one cosmetic, pharmaceutical and/or nutraceutical active ingredient is at least one selected from the group consisting of ceramides, sphingoid bases, sterols, peptides, amino acids, phenols, polyphenols, vitamins, extracts from plants or algae and cosmetic oils.

8. The composition according to claim 6, which comprises as component E) at least one ceramide and cholesterol.

9. The composition according to claim 1 which comprises
A) in an amount of from 0.1 wt.-% to 60 wt.-%,
B) in an amount of from 0.1 wt.-% to 60 wt.-%,
C) in an amount of from 10 wt to 95 wt.-%, as may be the case
D) in an amount of from 0.1 wt.-% to 80 wt-% and optionally further comprises
E) at least one cosmetic pharmaceutical and/or nutraceutical active ingredient in an amount of from 0.05 wt.-% to 20 wt.-%,
where the percentages by weight refer to the total composition.

10. The composition according to claim 1 wherein said liposomal composition has a mean particle size of 15 nm to 800 nm.

11. A formulation, comprising:
at least one liposomal composition according to claim 1.

12. The formulation according to claim 11, comprises
A) in an amount of from 0.0001 wt.-% to 6 wt.-%,
B) in an amount of from 0.0001 wt.-% to 6 wt.-%,
C) in an amount of from 20 wt.-% to 99 wt.-%, as may be the case
D) in an amount of from 0.0001 wt.-% to 30 wt.-%, and optionally further comprises
E) at least one cosmetic, pharmaceutical and/or nutraceutical active ingredient in an amount of from 0.00005 wt.-% to 40 wt.-%, wherein the percentages by weight refer to the total formulation.

13. A process for encapsulating at least one cosmetic, pharmaceutical and/or nutraceutical active ingredient comprising:
I) providing at least one phospholipid A), at least one biosurfactant B), at least one cosmetic, pharmaceutical and/or nutraceutical active ingredient E) and optionally at least one solvent which is at least one selected from the group consisting of alcohols comprising at least three carbon atoms and polyols D), and combining the above,
II) heating the provided components to a temperature in a range of from 50° C. to 100° C., and
III) heating water C) to a temperature in a range of from 50° C. to 100° C., and combining the water with the components provided in 1).

14. A capsule comprising:
at least one composition according to claim 1 for encapsulation of at least one cosmetic, pharmaceutical and/or nutraceutical active ingredient.

15. The composition according to claim 2, wherein the at least one phospholipid is a phosphatidylcholine.

16. The composition according to claim 3, wherein the at least one biosurfactant is a rhamnolipid.

17. The composition according to claim 4, wherein the at least one solvent is an alcohol which is at least one selected from the group consisting of alkanols and alkenols having 3 to 30 carbon atoms.

18. The composition according to claim 5, wherein the at least one solvent is glycerol.

19. The composition according to claim 6, wherein the at least one cosmetic, pharmaceutical and/or nutraceutical active ingredient has a water solubility at 25° C. and 1 bar of less than 1 mg/ml.

20. The composition according to claim 7, wherein the at least one cosmetic, pharmaceutical and/or nutraceutical active ingredient is selected from the group consisting of ceramides, sphingoid bases and sterols.
